(19) United States
(12) Patent Application Publication
(10) Pub. No.: US 2003/0096243 A1
(43) Pub. Date: May 22, 2003

(54) Methods and reagents for live-cell gene expression quantification

(76) Inventor: William Brian Busa, Renfrew, PA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/965,876

(22) Filed: Sep. 28, 2001

Related U.S. Application Data
(60) Provisional application No. 60/236,407, filed on Sep. 28, 2000.

(51) Int. Cl. C12Q 1/68; G06F 19/00; G01N 33/48; G01N 33/50

(52) U.S. Cl. 435/6; 702/20

(57) ABSTRACT

The present invention provides reagents and methods for mRNA quantification in intact cells involving providing cells that possess a target gene of interest that has been tagged with a binding site for an RNA binding protein, and a fluorescently labeled RNA binding polypeptide that includes an RNA binding domain that binds to the binding site, and calculating a quantity of expression of the target gene in the cells using fluorescence signaling techniques.

### CROSS REFERENCE

This application claims priority to U.S. Provisional Application Serial No. 60/236,407 filed Sep. 28, 2000.

### FIELD OF INVENTION

The invention relates to the fields of molecular biology, molecular genetics, and cellular biology.

### BACKGROUND

Quantification of gene expression is a field of intense interest in both basic biological research and in pharmaceutical drug discovery, since gene expression is a determinant of protein abundance and activity in cells, and most physiological cellular processes or pathological states are attributable to specific protein activities.

Commonly employed methods for quantifying the expression of a gene or genes in biological cells include manual and automated methods (employing so-called ‘gene chips,’ or cDNA microarrays) that all have numerous limitations in common, including

1. The requirement that cells be killed, and homogenized or broken open in order to extract the mRNA of interest;

2. The requirement for relatively large amounts of biological material, typically thousands to tens of thousands of cells per sample;

3. The involvement of one or more technically demanding or difficult steps that must be performed repeatedly in a rigorously quantitative manner, such as mRNA extraction, reverse transcription, and the polymerase chain reaction (PCR);

4. Poor signal-to-noise ratio. For example, quantification of gene expression via cDNA microarrays is widely regarded to be unreliable if the observed increment in expression is less than about two-fold.

The combination of these limitations tends to greatly restrict the temporal resolution of most gene expression studies, since achieving high temporal resolution would require numerous discrete samples comprising large amounts of biological material, as well as the precise performance of a very large number of technically demanding manipulations. This poor temporal resolution, in turn, negatively impacts the value of the data, since many clustering algorithms cannot meaningfully generate more clusters (in the analysis of a gene expression study) than the number of time points in the study, and gene expression changes of short duration or rapid time course are, of necessity, under-sampled.

The requirement for mRNA extraction from the cell, and thus cell lysis, prevents commonly employed techniques from reporting any information regarding the intracellular translocation and subcellular accumulation of the mRNAs of interest. It is now widely observed that a variety of mRNAs are not translated into proteins until they have been translocated to, and accumulate in, specific subcellular locations such as discrete regions of fertilized eggs, or bud tips in yeast cells (Bloom and Beach, 1999). Chemicals that block the translocation of specific mRNAs might prove to be effective and valuable drug candidates, but it is not straightforward to screen for such candidates today with common mRNA quantification techniques.

The requirement for large amount of biological material renders it difficult, if not impossible, to quantify the differing expression patterns in subpopulations of cells within a sample. By way of a simple example, if half the cells in a sample upregulate the expression of gene X four-fold, while the other half of the cells in the sample do not upregulate gene X's expression at all, all commonly employed quantification methods will report what appears to be a single response—a two-fold enhancement of expression by all the cells in the sample.

The necessity to employ technically demanding steps effectively prevents (or greatly complicates) the employment of gene expression quantification in a high-throughput mode (one in which tens or hundreds of thousands of observations per day are performed). High-throughput screening is today a staple of the pharmaceutical drug-discovery process, but high-throughput mRNA quantification has not yet proven practical.

Poor signal-to-noise ratios render common methods of mRNA quantification relatively insensitive. It is possible, perhaps even likely, that some gene expression up- and down-regulation events with magnitudes substantially less than two-fold are biologically significant events that today's methodologies cannot reliably detect.

In addition, a variety of techniques exist for indirectly detecting gene transcription by directly monitoring the abundance in cells of the protein that the gene of interest encodes. Some of these techniques, such as Western blotting, immunohistochemistry, and immunoprecipitation, suffer from the requirement that the cells must be killed and/or homogenized. Another such technique is live-cell fluorescence microscopy to detect chimeras of the protein of interest with a green fluorescent protein. (For example, see U.S. Pat. No. 6,203,986) All these techniques also suffer from the fact that they do not directly report gene transcription, but rather a consequence of gene transcription (namely, protein accumulation). As is known to those skilled in the art, protein accumulation in cells is affected by many factors other than gene transcription, such as proteolysis and secretion. Thus, measurement of protein abundance can be an unsatisfactory measure of gene transcription.

A method for observing and quantifying specific mRNA production and/or translocation via microscopy of live cells would avoid the limitations described above. Intact cells can be used, thus permitting high temporal resolution studies and enabling not only quantitation of mRNA abundance, but likewise quantitation of the mRNA's translocation to its effective subcellular locale. Microscopy also permits single-cell analysis, thus enabling the detection and quantification of differing responses in sub-populations of cells in a sample. Using image acquisition techniques to provide quantitation allows for relatively high-throughput mRNA quantitation studies via microscopy in intact cells. Finally, quantitation of many cellular responses via fluorescence microscopy routinely displays much better sensitivity than can be achieved with cDNA microarrays.

Various means have been reported for observation and/or quantification of specific mRNA synthesis via fluorescence microscopy of living cells, but these methods all suffer from drawbacks that make their routine application impractical in large-scale research. Sokol and colleagues (Sokol et al., 1998) quantified gene expression in live cells by employing ‘molecular beacons’, stem-loop oligodeoxynucleotides with matched donor and acceptor fluorophores on their 5′ and 3′ ends, which undergo fluorescence resonance energy transfer (FRET) when the molecular beacon is not bound to a complementary nucleic acid strand. Upon hybridizing with a target mRNA, FRET is inhibited, thus providing a quantifiable signal (reduction of FRET) that is proportional to the amount of mRNA bound by the reporter molecules and that thus may be proportional to the number of target mRNA molecules present in the cell. Sei-lida and coworkers (Sei-lida et al., 2000) and Tsuji et al. (2000) both employed a minor modification of this same approach, in which the donor and acceptor fluorophores were covalently attached to two separate oligodeoxynucleotides; side-by-side hybridization of the two oligos to a target mRNA yielded quantifiable FRET, which is not observed in the absence of the target mRNA.

All of these approaches suffer from the requirement that the fluorescent reporter molecules are synthetic molecules with little or no membrane permeability, and thus must be delivered to the cytoplasm via microinjection or other invasive and/or low-throughput means well-known to those skilled in the art (such as electroporation or biolistic delivery). Such means of delivering the reporter are time-consuming and often traumatic to the cells under study, severely restricting the number of cells that may be studied and the physiological conditions under which they may be studied. Furthermore, oligodeoxynucleotide probes that hybridize to the target mRNA's coding region may perturb the system under study by, for example, either blocking the translation of the target mRNA (thus potentially interfering in translation-dependent feedback loops that serve to regulate the target gene's expression) or by functioning as antisense oligonucleotides (triggering the destruction of the target mRNA), thus possibly altering the very parameter they are employed to measure (i.e., the target mRNA's abundance).

Thus, improved reagents and methods for specific mRNA quantification via fluorescence microscopy of intact cells are needed in the art.

### SUMMARY OF THE INVENTION

The present invention provides reagents and methods for mRNA quantification, wherein the methods comprise

(a) providing cells that possess:

(1) at least a first fluorescently labeled RNA binding polypeptide, wherein the first fluorescently labeled RNA binding polypeptide comprises a first RNA binding domain; and

(2) at least a first target gene of interest, where the target gene has been modified to comprise one or more nucleic acid sequences encoding a first binding site for the first RNA-binding domain, wherein upon expression of the first target gene into a first target RNA, the first binding site is specifically bound by the first fluorescently labeled RNA-binding polypeptide;

(b) scanning the cells to obtain fluorescent signals from the first fluorescently labeled RNA binding polypeptide;

(c) determining fluorescent emission intensities from the first fluorescently labeled RNA binding polypeptide at two different wavelengths;

(d) calculating a ratio of the fluorescent emission intensities from the first fluorescently labeled RNA binding polypeptide at the two different wavelengths; and

(e) calculating a quantity of the first target RNA in the cells from the ratio.

In a preferred embodiment, the fluorescently labeled RNA binding polypeptide further comprises a nuclear export signal. In another preferred embodiment, the fluorescently labeled RNA binding polypeptide is membrane permeant. In a further preferred embodiment, the first fluorescently labeled RNA binding polypeptide comprises a fluorophores pair selected from the group consisting of:

a) a donor/acceptor pair for fluorescence resonance energy transfer;

b) an excimer forming-pair; and

c) an exciplex-forming pair.

In another aspect, the invention provides reagents, and kits containing the reagents, comprising a fluorescently labeled RNA binding polypeptide, comprising:

(a) a non-naturally occurring amino acid sequence comprising

(i) a nuclear export signal; and

(ii) an RNA binding domain, wherein the amino acid; and

(b) a fluorophore pair selected from the group consisting of

(i) a donor/acceptor pair for fluorescence resonance energy transfer;

(ii) an excimer forming fluorophore pair; and

(iii) an exciplex forming fluorophore pair.

### BRIEF DESCRIPTION OF THE FIGURES

FIG. 1 represents a preferred embodiment of a procedure for quantifying target gene expression in response to some manipulation or treatment of the cells of interest.

FIG. 2 illustrates the normalized fluorescence emission spectra of 2.5 μM FITC-N₁₋₂₂-Rbod, collected at an excitation wavelength of 470 nm to excite the fluorescein donor.

FIG. 3 is a graph showing the titration of 2.5 μM FITC-N₁₋₂₂-Rhod with increasing concentrations of boxB RNA.

### DETAILED DESCRIPTION OF THE INVENTION

In one aspect, the present invention discloses a method for quantifying gene expression in live cells, comprising:

(1) at least a first fluorescently labeled RNA binding polypeptide, wherein the first fluorescently labeled RNA binding polypeptide comprises a first RNA binding domain; and

(2) at least a first target gene of interest, where the target gene has been modified to comprise one or more nucleic acid sequences encoding a first binding site for the first RNA-binding domain, wherein upon expression of the first target gene into a first target RNA, the first binding site is specifically bound by the first fluorescently labeled RNA-binding polypeptide;

(b) scanning the cells to obtain fluorescent signals from the first fluorescently labeled RNA binding polypeptide;

(c) determining fluorescent emission intensities from the first fluorescently labeled RNA binding polypeptide at two different wavelengths;

(d) calculating a ratio of the fluorescent emission intensities from the first fluorescently labeled RNA binding polypeptide at the two different wavelengths; and

(e) calculating a quantity of the first target RNA in the cells from the ratio.

The cells can be of any type, including but not limited to bacterial, yeast, and, preferably, mammalian cells.

As used herein, the term “gene expression” means transcription of the gene into an RNA copy.

As used herein, the term “scanning” means obtaining intensity measurements of the fluorescent signals from the fluorescently labeled RNA binding polypeptide. Such measurements can comprise either obtaining a spatial array of intensities or a single intensity measurement per field of view. In a preferred embodiment, the scanning comprises imaging the fluorescent signals from the fluorescently labeled RNA binding polypeptide, where “imaging” means obtaining a digital representation of the fluorescent signals from the fluorescently labeled RNA binding polypeptide, and does not require a specific arrangement or display of the digital representation. In preferred embodiments, well known formats for such “imaging” are employed, including but not limited to dib, tiff, jpg, .bmp. In further preferred embodiments, the images are displayed to provide a visual representation of the image.

As used herein the terms “quantity”, “quantitate”, and “quantifying” encompass both relative (i.e.: 2×, 4×, 0.2× the amount of RNA tag as in a control or other cell type being compared) and absolute (determining an actual concentration or amount of the RNA tag) measures of the amount of the target RNA.

The method of the invention can be used to quantitate the expression of any target gene, including expression of protein-encoding messenger RNA (mRNA) genes, ribosomal RNA encoding genes, and transfer RNA encoding genes , so long as the RNA expression product from the target gene possesses a sequence or structure (the “RNA tag”) that is bound specifically by the RNA binding polypeptide being used. In a preferred embodiment, the expression product of the target gene expression is a mRNA. Such RNA tags can comprise naturally occurring RNA binding sites for the RNA binding domain of the RNA binding polypeptide, or may comprise non-naturally occurring sites that have been selected based on their ability to bind to the RNA binding domain of the RNA binding polypeptide, using techniques known in the art, such as Systematic Evolution of Ligands by Exponential enrichment (SELEX), as described in U.S. Pat. No. 6,110,900.In a highly preferred embodiment, the RNA tag is a sequence that is not present or is very rare in the cells being analyzed.

The target gene of interest may be a gene native to the cell under study and present in the cell's genome, in which case the DNA sequence encoding the RNA tag may be inserted in or appended to the gene via techniques known to those skilled in the art, such as homologous recombination or retroviral insertion.

Alternatively, the target gene of interest may be one inserted into the genome by researchers employing molecular biological techniques such as retroviral insertion, in which case the DNA sequence encoding the RNA tag can be built into the gene prior to the gene's insertion in the genome by standard recombinant DNA techniques. In a further alternative, the target gene of interest may be contained in a plasmid used to stably or transiently transfect the cells under study, in which case again the DNA sequence encoding the RNA tag can be built into the target gene prior to transfection of the cells with the gene-containing plasmid. In a preferred embodiment, the RNA tag does not occur naturally in the cells under analysis.

Such tagging could also be achieved, for example, via homologous recombination to achieve site-directed tagging when the nucleotide sequence of the gene of interest is known. In homologous recombination, a single-stranded foreign DNA sequence may be inserted into an existing gene if the foreign sequence is flanked by nucleotide sequences identical to short (about 40 to 100 nucleotide) sequences that are adjacent in the gene into which the foreign sequence is to be inserted (Kucherlapati and Campbell, 1989). Such site-directed tagging offers the benefit of inserting the tag in a region of the gene that will cause minimal disruption of the gene and gene product functions. Preferably, the DNA encoding the RNA tag is inserted at a location in the gene of interest such that the RNA tag will be located in either the 3′ or 5′ untranslated region (UTR) of a mRNA transcribed from the gene. In these positions, the RNA tag will not alter the amino acid sequence of the protein translated from the tagged mRNA, thus avoiding perturbation of the protein's function, structure, localization, or abundance, nor is the tag likely to perturb the translation process itself by sterically hindering the ribosome entry site or the start site on the 5′ end of the mRNA. Indeed, it has been observed that yeast mRNAs bearing an MS2 coat protein-binding site in their 3′-UTRs, and with MS2 protein bound to these sites, have apparently normal half-lives and rates of poly-A tail de-adenylation (Wickens et al., 1999). If desired, multiple tagged genes can be produced in a single recombinant cell line.

If it is desired to quantify the expression of more than one target gene in the cell type (for example, to perform gene expression profiling), similar techniques may be employed to create a library of many different cell lines, each cell line in the library having a single, distinct tagged gene . Profiling of the expression of multiple genes may then be performed by growing and imaging the distinct cell lines in separate wells of a microplate, on separate domains of a miniaturized cell array (where each domain contains bound cells of a distinct cell line) (Taylor, 2000), by measuring fluorescence via a flow cytometer or, in general, by any means that allows the distinctly-tagged cell lines to be ‘addressed’ individually by the detection process. Such methods can be used in place of many current genomics and proteomics-based assays for determining gene expression profiles, as they can be conducted in a high throughput mode, and, since the assay utilizes intact cells, it provides data that is much more physiologically relevant than that provided by expression profiling of cDNA arrays, for example.

It may also be desirable to tag genes whose nucleotide sequences are not known. In such cases, one may employ undirected (e.g.:not site-specific) tagging via non-homologous recombination. By appropriately designing the nucleic acid sequences used for undirected tagging, a variety of techniques, such as restriction analysis, PCR, and cloning, may be employed to identify the gene that has been tagged, as well as the location of the tag within the gene.

The fluorescently labeled RNA binding polypeptides of the invention comprise RNA binding domains. These RNA binding domains can themselves be full length proteins with RNA binding activity, or fragments thereof that retain RNA binding activity, as well as synthetically derived polypeptide sequences that have been selected for their RNA binding activity, using techniques known in the art, such as Systematic Evolution of Ligands by Exponential enrichment (SELEX), as described in U.S. Pat. No. 6,110,900. The RNA binding polypeptide may be membrane permeant and added to the cell, or it may be encoded by an expression vector that is used to transfect the cells to be studied, thereby allowing expression of the RNA binding polypeptide by the cell.

The RNA-binding polypeptides may be fluorescently labeled via covalent attachment of appropriate fluorophores, as discussed below. In this case, it is preferred that the RNA binding polypeptide be membrane permeant, to permit loading of the cells with the RNA binding polypeptide simply by addition to the cell bathing medium. There are several classes of known membrane permeant peptides with RNA binding activity, including but not limited to arginine rich peptides (Tan and Frankel, 1995; Futaki et al., 2001). Furthermore, it is known that the addition of certain peptide sequences to other, non-membrane permeant polypeptides, results in a chimeric polypeptide that is membrane permeant. Such peptide sequences include, but are not limited to, peptides with 4-12 arginines; penetratin (RQIKIWFQNRRMKWKK) (SEQ ID NO: 1); signal sequence based peptides (GALFLGWLGAAGSTMGAWSQPKKKRKV (SEQ ID NO: 2); AAVALLPAVLLALLAP (SEQ ID NO:3); transportan (GWTLNSAGYLLKINLKALAALAKKIL) (SEQ ID NO:4); and amphiphilic model peptide (KLALKLALKALKAALKLA) (SEQ ID NO:5) (Futaki et al., 2001; Lindgen et al., 2000). Such membrane permeant polypeptides can be added to the cell at a wide range of concentrations; some arginine-rich peptides have shown no cytotoxicity when added to cells at up to 100 μM.

Alternatively, the RNA binding polypeptides may become fluorescently labeled by non-covalent binding to one or more fluorescent molecules. This alternative is especially useful for fluorescently labeling those RNA binding polypeptides that are not membrane permeant, and thus are expressed by the cell from an expression vector used to transfect the cell. In one such embodiment, the fluorescent molecule or molecules comprise synthetic, non-proteinaceous flourophores that are membrane-permeant, and thus diffuse into the cell when added to the bathing medium, and bind to the RNA binding polypeptide (see, for example, Griffin et al., 1998; Rozinov and Nolan, 1998). Specific binding of such a membrane-permeant fluorophore to the RNA-binding polypeptide may be achieved, for example, by adding an amino acid sequence to the RNA-binding polypeptide, (preferably outside the RNA-binding domain). for example, via ‘molecular evolution’ techniques to bind the membrane-permeant organic fluorophore with high affinity (for example, the fluorescein-binding antibody fragment described by Boder et al., 2000). In another embodiment, the fluorescent molecule or molecules comprise fluorescently-labeled synthetic peptides that are membrane-permeant (Lindgren et al, 2000), in which case a segment of said peptide is engineered to bind to the RNA-binding polypeptide, preferably outside the RNA-binding domain. The binding may occur at a site present in the native RNA-binding polypeptide, or else to an epitope engineered into the RNA-binding polypeptide. Engineering peptide-binding epitopes into the RNA-binding polypeptide may be advantageous when, for example, excimer or exciplex pair formation (see below) provides the readout, since this may enable highly precise positioning of the bound peptides, thus facilitating excimer or exciplex pair formation.

Various RNA binding domains are known in the art to bind with high specificity and affinity to distinct RNA sequences and/or structures. Examples of such RNA binding domain amino acid sequences include, but are not limited to, those shown in Table 1, together with their specific RNA tag. One of skill in the art will recognize that many other peptides with RNA binding domains can be utilized in the present invention, and that various modifications to the RNA binding domain amino acid sequence, as well as to the RNA tag sequence, can be prepared using standard techniques and verified to retain specific binding between the RNA binding domain and the RNA tag.

TABLE 1

| | | | |
|---|---|---|---|
| | RNA BINDING DOMAIN | 4 | |
| PEPTIDE | SEQUENCE | RNA TAG | REFERENCE |

| | | | |
|---|---|---|---|
| HIV rev (34- | TRQARRNRRRRWRERQR | GGUCUGGGCGCAGCGC | Tan and Frankel, |
| 50) | (SEQ ID NO:6) | AAGCUGACGGUACA | 1995; Tan et al., |
| | | (SEQ ID NO: 7) | 1993 |
| λN (1-22) | (M/L)DAQTRRRERRAEKQAQWK | NNGC(C/G)CUG(G/A)(G/ | Tan and Frankel, |
| | (SEQ ID NO:8) | A)(G/A)AAGGGCRR, | 1995; |
| | | wherein N is G or is absent | Chattopadhyay et |
| | | and R is C or is absent | al (1995) |
| | | (SEQ ID NO:9) | |
| P22N (14-30) | NAKTRRHERRRKLAIER | GGUGCGCUGACAAAGC | Tan and Frankel, |
| | (SEQ ID NO:10) | GCGCC (SEQ ID NO:11) | 1995 |
| HTLV-1 Rex | MPKTRRRPRRSQRKRP | GGGCGCCGGUACGCAA | Jiang et al., 1999 |
| peptide | (SEQ ID NO: 12) | GUACGACGGUACGCUC | |
| | | C (SEQ ID NO:13) | |
| HIV Tat (48- | GRKKRRQRRRPPQ | GGCCAGAUCUGAGCCU | Matsumoto et al., |
| 60) | (SEQ ID NO:14) | (SEQ ID NO:15) | 2000 |
| | | GGGAGCUCUCUGGCC | |
| | | (SEQ ID NO:16) | |

Other examples of proteins that can be utilized as RNA binding domains in the RNA binding polypeptides of the invention include, but are not limited to, the iron regulatory protein (IRP) (NCBI Protein database accession number AAF99861) which binds to a site in the 5′ untranslated region (5′-UTR) of ferritin mRNA (Gray et al., 1993); and bacteriophage MS2 coat protein (NCBI Protein database accession number AAA32260) (Bernardi et al., 1972)., which binds as a homodimer to a specific stem-loop structure in the viral replicase mRNA (ACAUGAGGAUUACCCAUGU (SEQ ID NO:17); or ACAUGAGGAUCACCCAUGU (SEQ ID NO: 18) (Valegard et al, 1997) that is rare or absent in the wild-type mRNAs of mammalian cell types.

In a preferred embodiment, the RNA binding polypeptide of the present invention further comprise a nuclear export sequence to ensure that the polypeptide does not accumulate in the nucleus, where it could interfere with appropriate RNA processing and subsequent export for translation. Such nuclear export sequences include, but are not limited to the nuclear export sequence from MEK1 (ALQKKLEELELDE) (SEQ ID NO:19) (Fukuda, (1997) J. Biol. Chem 272, 51, 32642-32648), MEK2 (DLQKKLEELELDE) (SEQ ID NO:20) (Zheng and Guan, J. Biol. Chem. 268:11435-11439, 1993), MAPKAP-2 (DKERWEDVKEEMTSALATMRVDYE) (SEQ ID NO:21) (Engel et al., 1998, EMBO J. 17:3363-3371), STATI (WDRTFSLFQQLLQSSFVVE) (SEQ ID NO:22) (Begitt et al., Proc. Natl. Acad. Sci. USA 97:10418-10423), HIV-1 REV (LPPLERLTL) (SEQ ID NO:23) (Mowen et al., 2000, Mol. Cell. Biol. 20:7273-7381), PKI (LALKLAGLDI) (SEQ ID NO:24) (Mowen et al., 2000, Mol. Cell. Biol. 20:7273-7381), I-kappa-B (LQQQLGQLTL) (SEQ ID NO:25) (Mowen et al., 2000, Mol. Cell. Biol. 20:7273-7381), c-Abl (LESNLRELQI) (SEQ ID NO:26) (Mowen et al., 2000, Mol. Cell. Biol. 20:7273-7381), Ahr (LDKLSVLTLS) (SEQ ID NO:27) (Ikuta et al., 1998, J. Biol. Chem. 273:2895-2904), Net (LWQFLLQLLLD) (SEQ ID NO:28) (Ducret, 1999, Mol. Cell. Biol. 19:7076-7087), cyclin BI (LCQAFSKVILA) (SEQ ID NO:29) (Ducret, 1999, Mol. Cell. Biol. 19:7076-7087), or other nuclear export sequences conforming to the NES consensus sequence XXXLXXLXL, where X is any amino acid (SEQ ID NO:30). In this embodiment, it is preferred that the nuclear export signal and the RNA binding domain are derived from different proteins. The order of the nuclear export signal and the RNA binding domain in the RNA binding polypeptide is not critical, and amino acid spacer sequences can separate the domains.

In the present method, the target gene is ‘tagged’ with a nucleotide sequence encoding the RNA binding domain's binding site (the “RNA tag”). Thus, where one of the RNA binding domains in Table 1 is to be used, sequences encoding one or more copies of the corresponding RNA tag would be engineered into the target gene of interest. Tagging with two adjacent copies of the RNA tag-encoding sequence is desirable when the readout depends upon the side-by-side binding of two RNA binding polypeptides, for example in some embodiments of FRET analysis, excimer, or exciplex pair formation analysis (see below).

A preferred RNA biding domain is derived from the bacteriophage λ N protein, which is used in conjunction with an RNA tag derived from the boxb RNA stem-loop structure in the N protein's own mRNA, to which the N protein binds specifically (Friedman and Court, 1995). A peptide comprising as little as the first 19 amino acids of N protein is capable of binding the boxB RNA stem-loop structure with high (nanomolar) affinity (Cilley and Williamson, 1997). The arginine-rich peptide comprising the first twenty-two amino acids of N protein (N₁₋₂₂) also binds boxb with high affinity, is predominantly in the random-coil conformation when free in solution (Tan and Frankel, 1995), assumes a fully alpha-helical conformation when bound to boxb (Legault et al., 1998), and (due to its arginine-rich sequence) has the characteristic of being a cell-penetrating peptide (Lindgren et al., 2000; Futaki et al., 2001). Thus, in this preferred embodiment, the domain from the bacteriophage λ N protein that can be used is selected from the group consisting of

| | |
|---|---|
| MDAQTRRRERRAEKQAQWKAANKG; | (SEQ ID NO:31) |
| MDAQTRRRERRAEKQAQWKAANK; | ((SEQ ID NO:32) |
| MDAQTRRRERRAEKQAQWK; | ((SEQ ID NO:33) |
| MDAQTRRRERRAEKQAQWKA; | (SEQ ID 34) |
| MDAQTRRRERRAEKQAQWKAA; | ((SEQ ID NO:35) |
| MDAQTRRRERRAEKQAQWKAAN; | (SEQ ID NO:36) |
| LDAQTRRRERRAEKQAQWKAANKG; | (SEQ ID NO:37) |
| LDAQTRRRERRAEKQAQWKAANK; | (SEQ ID 38) |
| LDAQTRRRERRAEKQAQWK; | (SEQ ID NO:39) |
| LDAQTRRRIERRAEKQAQWKA; | (SEQ ID NO:40) |
| LDAQTRRRERRAEKQAQWKAA; | (SEQ ID NO:41) |
| LDAQTRRRERLRAEKQAQWKAAN. | (SEQ ID NO:42) |

Furthermore, in this preferred embodiment the RNA tag comprises the nucleotide sequence NNGC(C/G)CUG(G/A)(G/A)(G/A)AAGGGCRR, wherein N is G or is absent and R is C or is absent (SEQ ID NO:9).

In carrying out the present invention, the cells can be cultured so that they express the target gene of interest (particularly for identifying compounds that inhibit gene expression), or so that they do not express the target gene (particularly for identifying compounds that promote gene expression).

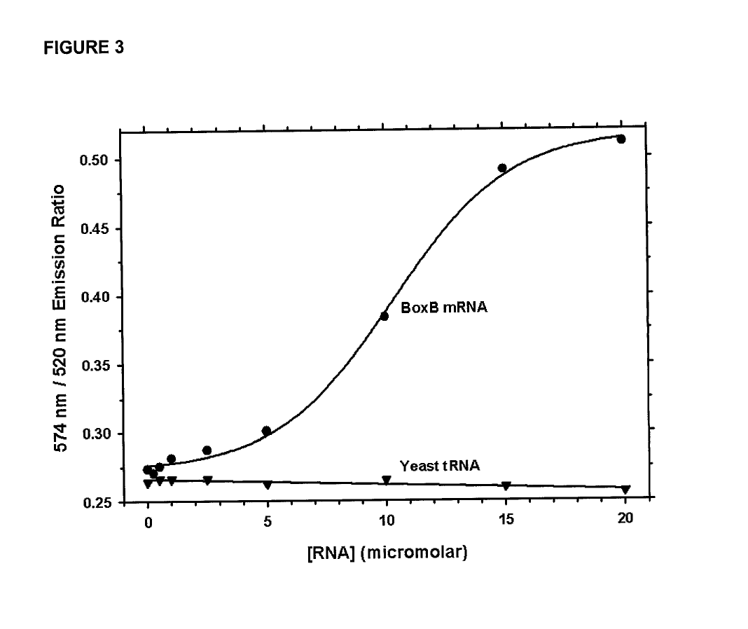

In practicing the invention, the cells are attached to or contained in an optically suitable surface or container and are examined via an optical system such as a fluorescence microscope, that incorporates an optical detector, such as CCD cameras, photomultiplier tubes, photodiodes, intensified cameras, and the like. Imaging detectors such as CCD cameras or intensified cameras are most useful where it is desirable to quantify expression levels for individual cells in a population. Non-imaging detectors such as photomultiplier tubes or photodiodes are useful when a population average measurement is sought. Numerous types of excitation light sources may be employed, such as lasers, arc lamps, and white light sources. If a white light source is employed for excitation, a filter wheel or similar device may be incorporated in the excitation path in order to, for instance, monitor more than one fluorophore, where each fluor excites at a different wavelength. Similarly, a filter wheel or similar device may be incorporated in the emission path in order to monitor more than one fluorophore, where each fluor emits at a different wavelength, and also to monitor the two distinct emission wavelengths of the RNA-binding peptide required in order to monitor FRET or excimer formation (as in FIG. 3). The digitized images from the detector, representing the fluorescence intensities of cells, are conveyed to a computer where software analyzes these images or signals and, with or without reference to a standard curve, automatically converts these intensity measurements to absolute or relative quantities of the target mRNA molecules, on either a per-unit-area or -volume, per-cell or per-image basis.

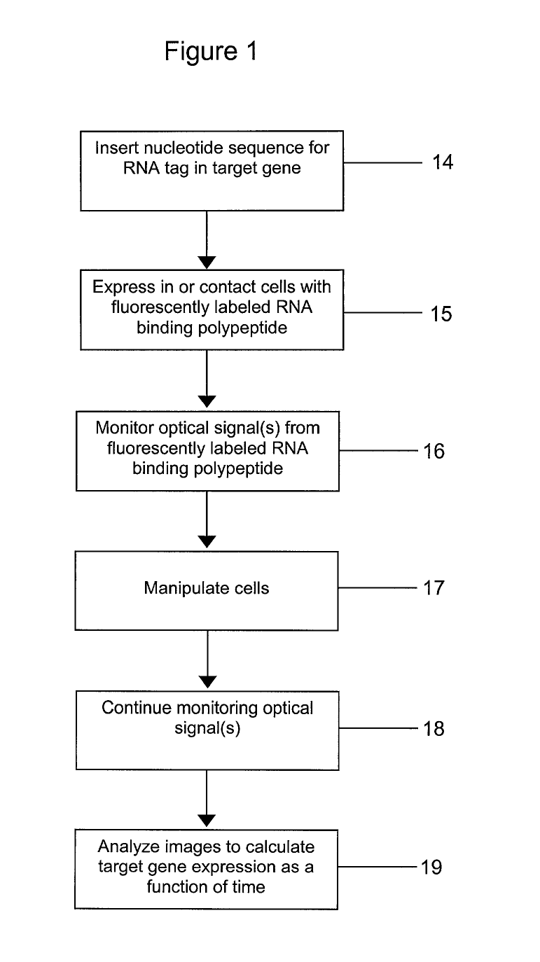

FIG. 1 represents a preferred embodiment of a procedure for quantifying target gene expression in response to some manipulation or treatment of the cells of interest. After modifying the target gene or genes of interest so that they encode mRNAs containing an RNA tag 14, the cells are contacted with (or induced to express) the fluorescently labeled RNA binding polypeptide 15, and are examined with a quantitative fluorescence microscopy or photometry system to collect a baseline value for expression of the target gene or genes 16. The cells are then manipulated as desired 17—this could involve, for instance, adding to the cell's bathing medium a drug, drug candidate, toxin, environmental sample, or biological molecule, but these are only exemplary manipulations. Following manipulation, the cells are further examined 18, a step which may be performed only once or else multiple times in order to collect a timecourse of gene transcription. Either subsequent to or simultaneous with this examination 18 the collected images are analyzed and target gene expression is quantified in either relative or absolute terms.

Alternatively, target gene expression in two distinct collections of cells may be compared, such as a comparison between the normal and cancerous forms of a cell type. Furthermore, timecourses of gene expression may be collected for a single collection of unmanipulated cells, such as cells in a developing embryo, or cells undergoing differentiation, cell division, growth, stasis, or other physiological processes.

In a preferred embodiment, quantitation of target gene expression is achieved via fluorescence microscopy. This readout may be achieved by any of a number of means. For example, the RNA binding polypeptide can be labeled such that the label provides one signal when the reporter molecule is bound to its target RNA and a different signal when not bound to its target, thus enabling quantification of the number of RNA binding polypeptides bound to the target RNA, and thus the quantity of target RNA expressed. This may be accomplished when a single molecule of the RNA binding polypeptide binds to the RNA tag. For example, a conformation change in the RNA binding polypeptide can alter the excitation or emission spectrum of a bound fluorophore, or can expose or hide a binding site on the RNA binding polypeptide for a second fluorescent molecule, whose fluorescence is altered upon binding the protein.

In a preferred embodiment, the RNA binding polypeptide can be labeled with two distinct fluorophores that serve as an efficient donor/acceptor pair for fluorescence resonance energy transfer (FRET) (Lakowicz, 1999, Chapter 13). The fluorophores need not be attached to the ends of the RNA binding polypeptide. For example, the fluorophores can be attached to the amino terminus of the polypeptide via a direct peptide bond; alternatively, the fluorophores may be linked to maleimide or iodoacetamide for attaching the fluorophore to a cysteine residue, or may be linked to isothiocyanate or succinimide ester for attaching the fluorophore to a lysine or the amino terminus of the polypeptide. The amino acid to which the fluorophore is attached is preferably unique within the polypeptide and can be placed anywhere within the polypeptide sequence, so long as its presence does not interfere with RNA binding, and (in embodiments in which the RNA-binding polypeptide is desired to be a membrane permeant peptide) so long as the peptide retains its ability to permeate the cell membrane.

The “donor” and “acceptor” fluorophores are typically selected as a matched pair wherein the absorption spectra of the acceptor molecule significantly overlaps the emission spectrum of the donor molecule. Preferably, the fluorescent donor and acceptor are selected such that both the absorption and the emission spectrum of the donor molecule is in the visible range (400 nm to about 700 nm), facilitating FRET detection in cells. The emission spectra, absorption spectra and chemical composition of many fluorophores are well known to those of skill in the art (see, for example, Handbook of Fluorescent Probes and Research Chemicals, R. P. Haugland, ed. which is incorporated herein by reference). The overlaps of many donor-acceptor pairs are listed in Wu and Brand, Resonance energy transfer: methods and applications. Analyt. Biochem, 1994. 218(1): p. 1-13. Preferred fluorophore pairs include, but are not limited to fluorescein or ALEXA FLUOR® 488 (donor)+rhodamine, eosin, erythrosin, QSY-7, ALEXA FLUOR® 546, BODIPY®-TMR Cy3, or ALEXA FLUOR® 532(acceptor); ALEXA FLUOR® 532 (donor)+ALEXA® 546 or rhodamine (acceptor); ALEXA FLUOR®350 (donor)+ALEXA FLUOR® 430 (acceptor); ALEXA FLUOR®430 (donor)+ALEXA FLUOR® 532, eosin, rhodamine, or Cy3 (acceptor). (Cy3 is available from Amersham Pharmacia; all others available from Molecular Probes, Eugene, Oreg.) Choice of an acceptor molecule depends on the above as well as availability of suitably reactive derivatives of the acceptor and its solubility (more soluble fluorophores are preferred). In a most preferred embodiment, the donor/acceptor pair is fluorescein and rhodamine.

The efficiency of excitation of the acceptor in a FRET pair by the donor is an extremely sensitive function of the distance between donor and acceptor, and the efficiency of FRET may be measured by exciting the donor and comparing the emission intensities of the donor and the acceptor. FRET can occur when the emission spectrum of a donor overlaps significantly the absorption spectrum of an acceptor molecule, and the donor and acceptor molecules are located within less than approximately 100 Angstroms of each other. (dos Remedios and Moens, 1995. J Struct Biol. 115:175-85; Emmanouilidou et al. 1999, Curr Biol. 9:915-918.)

In another preferred embodiment, the quantitative fluorescent readout may be achieved by other means, such as excimer or exciplex formation (Lakowicz, 1999, Chapter 1). Excimer formation involves formation of an excited state pairing of two molecules of the same fluorophore whose excitation and/or emission spectra differ greatly from those of the same fluorophore(s) when they are not interacting as a pair (The Photonics Dictionary, 42^{nd} International Edition, Laurin Publishing Co.), while exciplex formation involves formation of an excited state pairing of two different flurophores whose excitation and/or emission spectra differ greatly from those of the same fluorophore(s) when they are not interacting as a pair Excimer or exciplex formation can be achieved either between fluors labeling two or more amino acids of the same RNA binding polypeptide (and which are brought within the range required for excimer or exciplex formation by a polypeptide's conformation change upon binding to the RNA tag), or else between fluors on two or more separate RNA-binding polypeptides. In the latter case, the gene of interest can be tagged with two or more adjacent copies of the RNA tag. The adjacent binding of two or more labeled peptides to these two or more adjacent tags would then bring the fluors within the range required for excimer or exciplex formation, or for FRET analysis. Excimer and exciplex- pairs are well-known to be distinguished by fluorescence emission spectra substantially red-shifted from the emission spectra of the monomeric fluors. Thus, excimer or exciplex formation upon binding of the labeled peptide to the RNA tag may be measured by dividing the emission intensity of the excimer or exciplex by the emission intensity of the monomeric fluorophore, providing a quantitative measure of the amount of peptide bound to its RNA target. Excimer pair-forming fluorophores that can be used with the present methods include, but are not be limited to, pyrene and BODIPY-FL® (Molecular Probes, Eugene, Oreg.). Exciplex pair-forming fluorophores include, but are not limited to anthracene and diethylaniline (Molecular Probes, Eugene, Oreg.).

In examples where two RNA-binding polypeptides bind adjacently to the target RNA, the two RNA-binding polypeptides may be either identical RNA-binding polypeptides or else two distinct RNA-binding polypeptides that bind to distinct RNA tags engineered into the target RNA. The latter example may be preferable when the two fluorophores must interact in a precise spatial arrangement (as can be the case in excimer or exciplex pair formation). In any example in which binding of the RNA-binding polypeptides to the target RNA results in an alteration of the reporter(s) fluorescence spectra, it is preferred to quantify this binding by measuring the ratio of fluorescence excitation or emission at two distinct excitation or emission wavelengths.

Other fluorescence readout methods that can be used include, but are not limited to, methods in which the adjacent binding of two or more RNA-binding peptides to two or more adjacent RNA tags would (a) bring a fluorophore and a quencher of that fluorophore within effective range of each other to quench the fluorophore's fluorescence; (b) bring complementary fragments of an enzyme, such as dihydrofolate reductase, within range of each other, enabling those fragments to reform a functional enzyme that can either generate a colored or fluorescent molecule or bind a colored or fluorescent molecule (a so-called protein fragment complementation assay; Michnick et al., 2000); or (c) bring two fluorophores, constituting the donor and the acceptor of a FRET pair, within range of each other to achieve FRET.

In another embodiment of the invention the RNA-binding peptide is not supplied from outside the cell, but rather is expressed by the cell itself (which is either transiently or stably transfected to express the peptide, either constitutively or else under the control of an inducible promoter). In this embodiment, the application of synthetic organic fluorophores can be carried out as described above. Alternatively, the fluorophore(s) employed would be fluorescent proteins such as green fluorescent protein (GFP) or variants of a GFP, that are incorporated into the RNA-binding peptide by engineering a transfection construct encoding a fusion protein comprising the GFP and the RNA-binding polypeptide. The techniques for constructing and expressing fusion proteins are well known in the art (Sambrook et al.; 1989). In this embodiment, fluorescence detection would involve analysis of FRET between an appropriate GFP/GFP variant donor/acceptor pair, either engineered as a fusion protein in a single RNA binding polypeptide, or wherein the donor is expressed as a chimera with a first RNA binding polypeptide, and the acceptor is expressed as a chimera with a second RNA binding polypeptide, and wherein binding of the first and second RNA binding polypeptides to adjacent RNA tags in the target RNA brings the donor and acceptor into proximity to cause detectable alterations in FRET. The RNA binding polypeptides of the instant invention may be synthesized by any conventional method, including, but not limited to, those set forth in J. M. Stewart and J. D. Young, Solid Phase Peptide Synthesis, 2nd ed., Pierce Chemical Co., Rockford, Ill. (1984) and J. Meienhofer, Hormonal Proteins and Peptides, Vol. 2, Academic Press, New York, (1973) for solid phase synthesis and E. Schroder and K. Lubke, The Peptides, Vol. 1, Academic Press, New York, (1965) for solution synthesis. The disclosures of the foregoing treatises are incorporated by reference herein.

In general, these methods involve the sequential addition of protected amino acids to a growing peptide chain (U.S. Pat. No. 5,693,616, herein incorporated by reference in its entirety). Normally, either the amino or carboxyl group of the first amino acid and any reactive side chain group are protected. This protected amino acid is then either attached to an inert solid support, or utilized in solution, and the next amino acid in the sequence, also suitably protected, is added under conditions amenable to formation of the amide linkage. The fluorophores can be added during the solid-phase synthesis reaction, or as a later step in aqueous phase, as is known to those of skill in the art. After all the desired amino acids have been linked in the proper sequence, protecting groups and any solid support are removed to afford the crude polypeptide. The polypeptide is desalted and purified, preferably chromatographically, to yield the final product.

Preferably, peptides are synthesized according to standard solid-phase methodologies, such as may be performed on an Applied Biosystems Model 430A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art.

Alternatively, the RNA binding polypeptide can be produced via standard recombinant DNA technology. A DNA sequence encoding the desired amino acid sequence is cloned into an appropriate expression vector and used to transform a host cell so that the cell expresses the encoded peptide sequence. Methods of cloning, expression, and purification of recombinant peptides are well known to those of skill in the art. See, for example, Sambrook, et al., Molecular Cloning: A Laboratory Manual (2nd Ed., Vols. 1-3, Cold Spring Harbor Laboratory (1989)), Methods in Enzymology, Vol. 152: Guide to Molecular Cloning Techniques (Berger and Kimmel (eds.), San Diego: Academic Press, Inc. (1987)), or Current Protocols in Molecular Biology, (Ausubel, et al. (eds.), Greene Publishing and Wiley-Interscience, New York (1987). In this case, it is preferred to incorporate two unique amino acids to permit attachment of the two fluorophores, one on each end of the peptide, by the liquid phase methods.

After synthesis or recombinant production and isolation of the RNA binding polypeptide, the donor and acceptor fluorophores can be attached to the polypeptide by any of a number of means well known to those of skill in the art. In one embodiment, the fluorophores are linked directly from a reactive site on the fluorophore to a reactive group on the polypeptide such as a terminal amino or carboxyl group, or to a reactive group on an amino acid side chain such as a sulfhydryl, an amino, or a carboxyl moiety. Many fluorophores normally contain suitable reactive sites. Alternatively, the fluorophores may be derivatized to provide reactive sites for linkage to the RNA binding polypeptide. Suitable linkers are well known to those of skill in the art and include, but are not limited to, isothiocyanate, succinimide ester, maleimide, iodoacetamide, straight or branched-chain carbon linkers, heterocyclic carbon linkers, or peptide linkers. Such linkers can be used to simply increase spacing between the fluorophore(s) and the polypeptide, or to provide sites for functional interaction between the fluorophore(s) and the polypeptide. Fluorophores derivatized with functional groups for linking to a second molecule are commercially available from a variety of manufacturers. The derivatization may be by a simple substitution of a group on the fluorophore itself, or may be by conjugation to a linker.

Methods of quantifying gene expression products: In a preferred embodiment of the instant invention, binding of the fluorescently labeled RNA-binding polypeptides to the RNA tag yields a ratiometric readout; that is, a readout in which the ratio of the fluorophore's or fluorophores' emission intensities at two distinct wavelengths is a function of the fraction of the polypeptide molecules bound to RNA tags, and is thus also a function of the RNA tag's concentration. Ratiometric fluorescence measurements automatically correct for numerous artifacts that are otherwise problematic in quantitative fluorescence imaging of cells, including cell-to-cell variations in intracellular fluorophore concentration, variations in cell thickness (and thus variations in both fluorophore pathlength and cellular autofluorescence intensity) between different regions of a single cell, and variations in illumination intensity, optical collector efficiency, or photodetector efficiency across the field of view (Grynkiewicz et al., 1985). Such artifacts cancel out in a ratiometric measurement at two wavelengths because they contribute essentially equally to both the numerator and the denominator terms of the ratio. Peptides that exhibit alterations in FRET upon binding the RNA tag are well-suited to ratiometric measurement, a preferred ratio of interest being the emission intensity at or near the donor's emission maximum divided by the emission intensity at or near the acceptor's emission maximum (or vice versa). Similarly, peptides that exhibit alterations in excimer or exciplex formation are suitable for ratiometric measurements, by dividing the emission intensity characteristic of pure monomer, preferably at or near the monomer's emission maximum, by the emission intensity of the excimer or exciplex pair, preferably at or near the emission maximum characteristic of a pure excimer or exciplex pair (or vice versa). Peptides that alter the solvent exposure of a solvachromic fluorophore may be used in a ratiometric mode by dividing the emission intensity at or near the emission maximum of fully solvent-exposed fluorophore by the emission intensity at or near the emission maximum of the fully solvent-free fluorophore (or vice versa). Ratios thus calculated may be either single values (in the case of photometry) or else may be ratio images, or X/Y arrays of ratio values corresponding to the value of the ratio at each pixel of the images collected at each of the two emission wavelengths, particularly when using fluorescence microscopy employing an imaging detector such as a CCD camera.

Ratios determined as described above may be employed in a variety of ways to calculate relative or absolute changes in the tagged RNA's quantity. Most simply, the practitioner may relate the experimentally determined ratio to a previously determined calibration curve, determined by measuring ratios for a series of solutions each containing a fixed concentration of the RNA-binding polypeptide mixed with one of several known concentrations of the RNA tag (e.g., see FIG. 3). In such calibration solutions, the concentration of the RNA-binding polypeptide and the range of concentrations of RNA tag employed should be those expected to approximate their intracellular concentrations under the experimental conditions. Because the absolute values of fluorescence ratios are instrument-dependent, the data for such a calibration curve is preferably collected on the same instrument with which the experimental measurements will be collected. Ordinarily, the calibration curve need not be collected using living cells; on a microscope, droplets of peptide/RNA solution may be imaged, as may thin layers of RNA binding polypeptide/RNA tag solution, provided the ionic composition of the calibration solutions are not so different from cytosolic conditions as to significantly alter the RNA binding polypeptide's affinity for the RNA tag. In a spectrofluorometer, solutions contained in cuvettes may be used. Ionic compositions approximating those of cytosol are well known to those skilled in the art.

Under some circumstances, such as when it is difficult or impossible to repeatedly construct multi-point calibration curves due to limited availability of purified tagged RNA, it may be desirable and practical to mathematically derive a calibration curve based only on measurements of the peptide's ratio in the absence of the RNA tag and in the presence of a saturating concentration of the RNA tag, plus the peptide/RNA dissociation constant. One such mathematical derivation has been previously discussed in Grynkiewicz et al., 1985, for the case of a ratiometric indicator for intracellular Ca2+ concentration. Adapting the Grynkiewicz formula to determine a tagged RNA's concentration employing the instant invention, we have:

[RNA]=K_{d}*((R−Rₘᵢₙ)/(Rₘₐₓ−R))*(S_{f2}/S_{b2}); where

[RNA]=the RNA tag concentration,

K_{d}=the dissociation constant of the RNA tag/RNA binding polypeptide complex

R=the experimentally observed ratio of the two emission intensities at wavelengths 1 and 2 (=I₁/I₂)

Rₘᵢₙ=the ratio of the two emission intensities in the absence of RNA tag (=Iₘᵢₙ₁/Iₘᵢₙ₂)

Rₘₐₓ=the ratio of the two emission intensities in the presence of a saturating concentration of RNA tag (=Iₘₐₓ₁/Iₘₐₓ₂)

S_{f2}=the emission intensity at the denominator wavelength, in the absence of RNA tag (=Iₘᵢₙ₂)

S_{b2}=the emission intensity at the denominator wavelength, in the presence of a saturating concentration of RNA tag (=Iₘₐₓ₂)

In another embodiment, the methods further comprise determining the localization of the fluorescently labeled RNA binding polypeptide, and thus the target RNA. For examples, the cells could be further contacted with a fluorescent reporter molecule that reports nuclear location, in order to identify individual cells. The location of the fluorescent signals from the fluorescently labeled RNA binding polypeptide can then be determined within the individual cells by various means, such as those disclosed in U.S. Pat. No. 5,989,835.

The present invention further provides novel fluorescently labeled RNA binding polypeptides, comprising:

(a) an amino acid sequence comprising

(i) a nuclear export signal; and

(ii) an RNA binding domain; and

(b) a fluorophore pair selected from the group consisting of

(i) a donor/acceptor pair for fluorescence resonance energy transfer;

(ii) an excimer forming fluorophore pair; and

(iii) an exciplex forming fluorophore pair

The order of the nuclear export signal and the RNA binding domain in the RNA binding polypeptide is not critical, and amino acid spacer sequences can separate the domains. The details for fluorophore attachment to the reagents is as described above.

In a preferred embodiment, the amino acid sequence is non-naturally occurring. As used herein, the phrase “non-naturally occurring” means that the nuclear export signal and the RNA binding domain are not derived from the same protein, but represent modules from different proteins, or modules derived synthetically using techniques such as molecular evolution.

In a preferred embodiment, the fluorescently labeled RNA binding polypeptide is membrane permeant. In a further preferred embodiment, the nuclear export signal comprises an amino acid sequence of the general formula

XXXLXXLXL, where X is any amino acid (SEQ ID NO:30), including but not limited to the following nuclear export sequences: ALQKKLEELELDE (SEQ ID NO:19); DLQKKLEELELDE (SEQ ID NO:20); LPPLERLTL (SEQ ID NO:23); LQQQLGQLTL (SEQ ID NO:25); LDKLSVLTLS (SEQ ID NO:27); and LWQFLLQLLLD (SEQ ID NO:28).

In a further embodiment, the nuclear export signal comprises an amino acid sequence selected from the group consisting of: DKERWEDVKEEMTSALATMRVDYE (SEQ ID NO:21); WDRTFSLFQQLLQSSFVVE (SEQ ID NO:22); LALKLAGLDI (SEQ ID NO:24); LESNLRELQI (SEQ ID NO:26); and LCQAFSKVILA (SEQ ID NO:29).

In a further embodiment, the RNA binding domain of the novel fluorescently labeled RNA binging polypeptides comprises an amino acid sequence selected from the group consisting of: TRQARRNRRRWRERQR (SEQ ID NO:6); (M/L)DAQTRRRERRAEKQAQWK (SEQ ID NO:8); NAKTRRHERRRKLAIER (SEQ ID NO:10); MPKTRRRPRRSQRKRP (SEQ ID NO:12); and GRKKRRQRRRPPQ (SEQ ID NO:14).

In a further embodiment, the fluorophore pair is a donor/acceptor pair for fluorescence resonance energy transfer. In a most preferred version of this embodiment, the donor/acceptor pair is selected from the group consisting of

fluorescein (d)+rhodamine(a)

fluorescein (d)+eosin (a)

fluorescein (d)+erythrosine (a)

fluorescein (d)+QSY-7 (a)

fluorescein (d)+ALEXA FLUOR® 54 (a)

fluorescein (d)+BODIPY®-TMR Cy3 (a)

fluorescein (d)+ALEXA FLUOR® 532 (a)

ALEXA FLUOR® 488 (d)+rhodamine (a)

ALEXA FLUOR® 488 (d)+eosin (a)

ALEXA FLUOR® 488 (d)+erythrosine (a)

ALEXA FLUOR® 488 (d)+QSY-7 (a)

ALEXA FLUOR® 488 (d)+ALEXA FLUOR® 54 (a)

ALEXA FLUOR® 488 (d)+BODIPY®-TMR Cy3 (a)

ALEXA FLUOR® 488 (d)+ALEXA FLUOR® 532 (a)

ALEXA FLUOR® 532 (d)+ALEXA FLUOR® 546 (a)

ALEXA FLUOR® 532 (d)+rhodamine (a)

ALEXA FLUOR® 350 (d)+ALEXA FLUOR® 430 (a);

ALEXA FLUOR® 430 (d)+ALEXA FLUOR® 532 (a)

ALEXA FLUOR® 430 (d)+eosin (a)

ALEXA FLUOR® 430 (d)+rhodamine (a)

ALEXA FLUOR® 430 (d)+BODIPY®-TMR Cy3 (a)

In a further embodiment, the fluorophore pair is an excimer-forming pair. In a most preferred version of this embodiment, the excimer-forming pair is selected from the group consisting of a pyrene pair; and a BODIPY-FL® pair. In a further embodiment, the fluorophore pair is an exciplex-forming pair. In a most preferred version of this embodiment, the exciplex-forming pair consists of anthracene and diethylaniline.

In a further embodiment, the fluorescently labeled RNA binding polypeptide further comprises an amino acid sequence to impart membrane permeability on the polypeptide, including but not limited to an amino acid sequence selected from the group consisting of

| | |
|---|---|
| RQIKIWFQNRRMKWKK; | (SEQ ID NO:1) |
| GALFLGWLGAAGSTMGAWSQPKKKRKV; | (SEQ ID NO:2) |
| AAVALLPAVLLALLAP; | (SEQ ID NO:3) |
| GWTLNSAGYLLKINLKALAALAKKIL; | (SEQ ID NO:4) |
| KLALKLALKALKAALKLA; and | (SEQ ID NO:5) |

amino acid sequences of between 4 and 30 amino acids comprising between 4 and 12 arginine residues.

In a further embodiment, the fluorescently labeled RNA binding polypeptide comprises:

(a) an RNA binding domain consisting of an amino acid sequence selected from the group consisting of:

| | |
|---|---|
| MDAQTRRRERRAEKQAQWKAANKG; | (SEQ ID NO:31) |
| MDAQTRRRERRAEKQAQWKAANK; | (SEQ ID NO:32) |
| MDAQTRRRERRAEKQAQWK; | (SEQ ID NO:33) |
| MDAQTRRRERRAEKQAQWKA; | (SEQ ID 34) |
| MDAQTRRRERRAEKQAQWKAA; | (SEQ ID 35) |
| MDAQTRRRERRAEKQAQWKAAN; | (SEQ ID 36) |
| LDAQTRRRERRAEKQAQWKAANKG; | (SEQ ID 37) |
| LDAQTRRRERRAEKQAQWKAANK; | (SEQ ID 38) |
| LDAQTRRRERRAEKQAQWK; | (SEQ ID 39) |
| LDAQTRRRERRAEKQAQWKA; | (SEQ ID 40) |
| LDAQTRRRERRAEKQAQWKAA; and | (SEQ ID 41) |
| LDAQTRRRERRAEKQAQWKAAN; and | (SEQ ID 42) |

(b) a donor/acceptor fluorophore pair selected from the group consisting of:

fluorescein/rhodamine;

fluorescein/eosin;

fluorescein/erythrosine;

fluorescein/QSY-7;

fluorescein/ALEXA FLUOR® 54;

fluorescein/BODIPY®-TMR Cy3;

fluorescein/ALEXA FLUOR® 532;

ALEXA FLUOR® 488/rhodamine;

ALEXA FLUOR® 488/eosin;

ALEXA FLUOR® 488/erythrosine;

ALEXA FLUOR® 488/QSY-7;

ALEXA FLUOR® 488/ALEXA FLUOR® 54;

ALEXA FLUOR® 488/ BODIPY®-TMR Cy3;

ALEXA FLUOR® 488/ALEXA FLUOR® 532;

ALEXA FLUOR® 532 /ALEXA FLUOR® 546;

ALEXA FLUOR® 532/rhodamine;

ALEXA FLUOR® 350/ALEXA FLUOR® 430;

ALEXA FLUOR® 430/ALEXA FLUOR® 532;

ALEXA FLUOR® 430/eosin;

ALEXA FLUOR® 430/rhodamine; and

ALEXA FLUOR® 430/BODIPY®-TMR Cy3.

In a still further embodiment, the fluorescently labeled RNA binding polypeptide comprising:

(a) an RNA binding domain consisting of an amino acid sequence selected from the group consisting of:

| | |
|---|---|
| MDAQTRRRERRAEKQAQWKAANKG; | (SEQ ID NO:31) |
| MDAQTRRRERRAEKQAQWKAANK; | (SEQ ID NO:32) |
| MDAQTRRRERRAEKQAQWK; | (SEQ ID NO:33) |
| MDAQTRRRERRAEKQAQWKA; | (SEQ ID 34) |
| MDAQTRRRERRAEKQAQWKAA; | (SEQ ID 35) |
| MDAQTRRRERRAEKQAQWKAAN; | (SEQ ID 36) |
| LDAQTRRRERRAEKQAQWKAANKG; | (SEQ ID 37) |
| LDAQTRRRERRAEKQAQWKAANK; | (SEQ ID 38) |
| LDAQTRRRERRAEKQAQWK; | (SEQ ID 39) |
| LDAQTRRRERRAEKQAQWKA; | (SEQ ID 40) |
| LDAQTRRRERRAEKQAQWKAA; and | (SEQ ID 41) |
| LDAQTRRRERRAEKQAQWKAAN; and | (SEQ ID 42) |

(b) a nuclear export signal consisting of an amino acid selected from the group consisting of:

(SEQ ID NO:30)

| | |
|---|---|
| (i) | XXXLXXLXL, where X is any amino |
| acid; | |

(SEQ ID NO:19)

| | |
|---|---|
| (ii) | ALQKKLEELELDE; |

(SEQ ID NO:20)

| | |
|---|---|
| (iii) | DLQKKLEELELDE; |

(SEQ ID NO:23)

| | |
|---|---|
| (iv) | LPPLERLTL; |

(SEQ ID NO:25)

| | |
|---|---|
| (v) | LQQQLGQLTL; |

(SEQ ID NO:27)

| | |
|---|---|
| (vi) | LDKLSVLTLS; |

(SEQ ID NO:28)

| | |
|---|---|
| (vii) | LWQFLLQLLLD; |

(SEQ ID NO:21)

| | |
|---|---|
| (viii) | DKLERWEDVKEEMTSALATMRVDYE; |

(SEQ ID NO:22)

| | |
|---|---|
| (ix) | WDRTFSLFQQLLQSSFVVE; |

(SEQ ID NO:24)

| | |
|---|---|
| (x) | LALKLAGLDI; |

(SEQ ID NO:26)

| | |
|---|---|
| (xi) | LESNLRELQI; and |

(SEQ ID NO:29)

| | |
|---|---|
| (xii) | LCQAFSKVILA; and |

(c) a donor/acceptor fluorophore pair selected from the group consisting of:

fluorescein/rhodamine;

fluorescein/eosin;

fluorescein/erythrosine;

fluorescein/QSY-7;

fluorescein/ALEXA FLUOR® 54;

fluorescein/BODIPY®-TMR Cy3;

fluorescein/ALEXA FLUOR® 532;

ALEXA FLUOR® 488/rhodamine;

ALEXA FLUOR® 488/eosin;

ALEXA FLUOR® 488/erythrosine;

ALEXA FLUOR® 488/QSY-7;

ALEXA FLUOR® 488/ALEXA FLUOR® 54;

ALEXA FLUOR® 488/ BODIPY®-TMR Cy3;

ALEXA FLUOR® 488/ALEXA FLUOR® 532;

ALEXA FLUOR® 532 /ALEXA FLUOR® 546;

ALEXA FLUOR® 532/rhodamine;

ALEXA FLUOR® 350/ALEXA FLUOR® 430;

ALEXA FLUOR® 430/ALEXA FLUOR® 532;

ALEXA FLUOR® 430/eosin;

ALEXA FLUOR® 430/rhodamine; and

ALEXA FLUOR® 430/BODIPY®-TMR Cy3.

In another aspect of the present invention, the present invention provides kits for carrying out the invention, wherein the kits contains one or more of the fluorescently labeled RNA binding polypeptides of the invention together with instructions for their use in the methods of the invention. In a further embodiment, the kits also contain a vector containing the DNA sequence encoding an RNA tag to be used in conjunction with the fluorescently labeled RNA binding polypeptides to carry out the methods of the invention.

The invention is illustrated by the following example of the construction of a fluorescently-labeled RNA-binding polypeptide that yields a quantifiable signal upon binding to its RNA tag, the use of this peptide in fluorescence photometry to quantify the concentration of an RNA tag, and the delivery of this polypeptide into cells as a cell-penetrating peptide. This example is provided for the purpose of illustration only, and should not be construed as limiting.

### EXAMPLES

N₁₋₂₂ peptide, modified for convenient synthesis to contain an additional C-terminal glycine, (LDAQTRRRERRAEKQAQWKAANKG-OH (SEQ ID NO: 31) was synthesized and doubly-labeled with fluorescein isothiocyanate (FITC) and rhodamine (Rhod) to yield the reagent FITC-N₁₋₂₂-Rhod, wherein the FITC label is attached to the amino terminus of the peptide and the rhodamine label is conjugated to the lysine residue near the carboxy terminus. This peptide was dissolved in 50 mM HEPES, pH 7.2 to a concentration of 0.5 mM as a stock solution. boxB RNA, 5′ GGGCCCUGAAAAAGGGCCC (SEQ ID NO: 43), was synthesized and dissolved in RNAsecure solution (Ambion, Inc.), at a concentration of 100 μM as a stock solution. boxB bases that are either crucial or irrelevant to its interaction with N protein have been identified by Chattopadhyay et al (1995) via base substitution. For example, the point-mutant boxB sequence GCCCUAAAAAAGGGC (SEQ ID NO: 44) displays less than 1% of the in vivo activity of the native sequence, GCCCUGAAAAAGGGC (SEQ ID NO: 45), whereas the point-mutant GCCCUAGAAAAGGGC (SEQ ID NO: 46) maintains nearly 90% of the activity of the native sequence. Point mutants of the boxB sequence that do not unacceptably reduce boxB binding are also encompassed by the instant invention. Yeast tRNA (Sigma-Aldrich Inc.) was dissolved in RNAsecure solution to a concentration equaling 250 OD units (measured at 260 nm) per ml. All experiments reported here were performed by diluting these stock solutions to the specified final concentrations in a buffer composed of 10 mM HEPES, pH 7.2, 100 mM KCl, 1 mM MgCl₂, 0.5 mM EDTA.

The N₁₋₂₂ polypeptide binds boxB with high affinity, is predominantly in the random-coil conformation when free in solution, and assumes a fully alpha-helical conformation when bound to boxB, as discussed above. The root-mean-square distance between the two termini of a peptide is greater when that peptide is in the random-coil conformation than when it is in the alpha-helical conformation and thus the efficiency of FRET between donor and acceptor at the two termini of N₁₋₂₂ will increase when the labeled peptide binds to the RNA tag and assumes an alpha-helical conformation. This may be measured by dividing the peak emission intensity of the acceptor by the peak emission intensity of the donor, providing a quantitative measure of the amount of peptide bound to its RNA target.

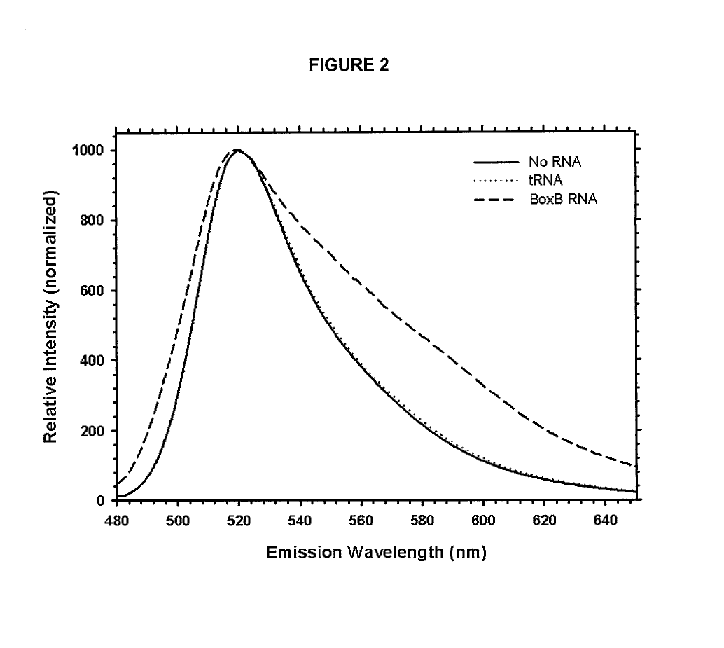

FIG. 2 illustrates the normalized fluorescence emission spectra of 2.5 μM FITC-N₁₋₂₂-Rhod, collected at an excitation wavelength of 470 nm to excite the fluorescein donor. In the absence of RNA, a typical fluorescein emission spectrum is observed when fluorescein is directly excited. In the presence of an irrelevant RNA (yeast tRNA, at a concentration yielding an OD₂₆₀ equivalent to 2.5 μM boxB RNA), an essentially identical spectrum is observed. In contrast, in the presence of 2.5 μM boxB RNA a broad shoulder is observed, centered at approximately 574 mn, indicating FRET occurring between the fluorescein donor and the rhodamine acceptor upon binding of FITC-N₁₋₂₂-Rhod to boxB RNA.

The ratio of FITC-N₁₋₂₂-Rhod emissions at 574 and 520 nm provides a quantitative measure of boxB RNA concentration. In the experiment of FIG. 3, titration of 2.5 μM FITC-N₁₋₂₂-Rhod with increasing concentrations of boxb RNA yields a sigmoidal curve typical of macromolecular binding, with the 574/520 ratio approximately doubling over the RNA concentration range 0 to 20 μM. In contrast, irrelevant RNA (yeast tRNA) elicits no increase in the 574/520 ratio over this same concentration range.

The amino acid sequence of N₁₋₂₂ contains 5 arginines, making this an arginine-rich peptide that may be expected to act as a cell-penetrating peptide capable of passively diffusing across plasma membranes into cells. To confirm this, Hela cells were grown in microtiter plates in EMEM+10% fetal bovine serum. Experimental cells were then exposed to 20 μM FITC-N₁₋₂₂-Rhod in this same medium for 1 h at 37° C.; control cells were treated with culture medium without FITC-N₁₋₂₂-Rhod. Following incubation, cells were washed once with phosphate buffered saline, once with EMEM+10% fetal bovine serum, and once more with phosphate buffered saline, then immediately imaged on an inverted epifluorescence microscope with a CCD camera and a 20× objective, employing a standard rhodamine filter set. The image data demonstrated that doubly-labeled FITC-N₁₋₂₂-Rhod was cell-penetrating: cells treated with FITC-N₁₋₂₂-Rhod were fluorescent throughout their cytoplasm and thus readily visualized, whereas control cells were non-fluorescent under the same imaging conditions.

Prophetic Example:

Given a cell-permeant, ratiometric, fluorescent, specific RNA-binding polypeptide such as FITC-N₁₋₂₂-Rhod, and an RNA tag to which it binds specifically such as the boxB sequence, the instant invention may be practiced in the following manner to compare the level of expression of a known gene ‘X’ among several samples of cells, each sample contacted with one member of a library of candidate compounds in an effort to identify a compound that alters (in this example, inhibits) the transcription of gene X. This application of the invention, and this sequence of steps to achieve it, are provided for purposes of illustration only and should not be viewed as limiting.

Tagging the Gene of Interest:

Gene X is a known gene, i.e., one whose DNA sequence is known from either public or proprietary sources. A suitable cell line that expresses gene X (either constitutively or inducibly) is selected, if one exists. If such a cell line is not known to exist, one is engineered by transfecting an existing cell line with a plasmid containing gene X under the control of the relevant promoter or promoters. If an existing cell line that expresses a chromosomal copy of gene X is selected, that chromosomal gene is tagged in the region encoding the 3′ untranslated region of its cognate RNA with the DNA sequence encoding the boxB RNA sequence. Such tagging may be achieved via in vivo homologous sequence targeting, as described in U.S. Pat. No. 5,763,240. If a cell line is engineered to express gene X via transfection with a plasmid, the copy of gene X inserted into the plasmid may be engineered to contain in the region encoding the 3′ untranslated region of its cognate RNA the DNA sequence encoding the boxB RNA sequence. In either case, cells successfully tagged via homologous recombination or via transfection are then selected by means known to those skilled in the art, and are expanded to establish at least one clone of properly tagged cells (referred to hereafter as ‘the tagged cell line’).

Cells of the tagged cell line are grown to a convenient density in the wells of 96-well microtiter plates and, if necessary, are induced to express gene X. The cells of each well are then contacted with FITC-N₁₋₂₂-Rhod at a suitable concentration (for example, 2.5 microM) for a suitable period (for example, 1 hour) to achieve intracellular loading sufficient for fluorescence microscopy. The cells are then washed several times with culture medium not containing FITC-N₁₋₂₂-Rhod in order to remove extracellular peptide. The precise point at which peptide loading is performed is determined empirically to achieve adequate loading without allowing time for significant intracellular degradation of the peptide.

The cells of each well are then contacted with one compound from a library of compounds which, it is hoped, contains at least one compound that inhibits the transcription of gene X. After a suitable fixed incubation time, each well is imaged via an inverted epifluorescence microscope, at an excitation wavelength of 470 nm, and for each well two images are collected with a CCD camera under otherwise identical conditions at emission wavelengths of 574 nm and 520 nm. Employing a computer, the pairs of images are used to construct a ratio image for each well (by pixel-by-pixel division of the 574 nm image intensity by the 520 nm image intensity). From this ratio image, the per-cell or per-well average ratio for each well is determined.

One or more wells are employed as controls by contacting their cells with FITC-N₁₋₂₂-Rhod but not contacting their cells with library compounds. From ratio images of these wells, the natural variation in the 574/520 ratio is determined, and may be expressed, for example, as the standard deviation of the mean of several control wells.

By referring to the control data, the researcher selects a maximum value for the 574/520 ratio that he considers a ‘hit’ (a level indicative of inhibition of gene X transcription). In this example, a hit is considered to be indicated by any 574/520 ratio value that is more than 1 standard deviation below the controls' mean. The researcher then examines the ratios for the experimental wells, identifying those which constitute hits. The library compounds with which those wells were contacted may then be considered candidate compounds inhibiting the transcription of gene X.

Because the 574/520 ratio is not a linear function of RNA concentration (see FIG. 3), it may be desired to convert 574/520 ratios to estimated RNA concentrations either before or after designating hits. This is done by reference to a previously-constructed calibration curve, such as that of FIG. 3.

### REFERENCES

Beach D L, Salmon E D, and Bloom K (1999). Localization and anchoring of mRNA in budding yeast. Curr. Biol. 9:569.

Bernardi A, and Spahr P F (1972). Proc. Natl. Acad. Sci USA 69:3033.

Bertrand E, Chartrand P, Schaefer M, Shenoy SM, Singer R H, and Long R M (1998). Localization of ASH1 mRNA particles in living yeast. Mol. Cell 2:437.

Bloom K, and Beach, D L (1999). mRNA localization: motile RNA, asymmetric anchors. Curr. Opinion Microbiol. 2:604-609.

Boder, E T, Midelfort K S, Wittrup K D (2000). Directed evolution of antibody fragments with monovalent femtomolar antigen-binding affinity. Proc Natl Acad Sci U S A 97:10679.

Chattopadhyay S, Garcia-Mena J, DeVito J, Wolska K, and Das A (1995). Bipartite function of a small RNA hairpin in transcription antitermination in bacteriophage lambda. Proc. Natl. Acad. Sci. USA 92:4061.

Cilley and Williamson RNA 3(1):57-67 (1997).

Chu E, Voeller D, Koeller D M, Drake J C, Takimoto C H, Maley G F, Maley F, and Allegra C J (1993). Identification of an RNA binding site for human thimidylate synthase. Proc. Natl. Acad. Sci USA 90:517.

Friedman and Court, Mol. Microbiol. 1995 Oct; 18(2):191-200.

Futaki et al., J. Biol. Chem. 276:5836-5840 (2001).

Gray N K, Quick S, Goossen B, Constable A, Hirling H, Kuhn L, and Hentze M W (1993). Recombinant iron regulatory factor functions as an IRE-binding protein, a translational repressor and an aconitase. A functional assay for translational repression and direct demonstration of the iron switch. Eur. J. Biochem. 18:657.

Griffin B A, Adams S R, and Tsien R Y (1998). Specific covalent labeling of recombinant protein molecules inside living cells. Science 281:269.

Grynkiewicz G, Poenie M, and Tsien R Y (1985). A new generation of Ca2+ indicators with greatly improved fluorescence properties. J. Biol. Chem. 260:3440.

Jiang et al., Structure Fold Des. 1999 Dec. 15; 7(12):1461-72.

Kucherlapati R S and Campbell C (1989). Single-stranded site-directed modification of mammalian genes in vivo. PCT Patent number WO 90/14092.

Legault et al., Cell 93:289-299 (1998).

Lindgren M, Hallbrink M, Prochiantz A, and Langel U (2000). Cell-penetrating peptides. Trends Pharmacol. Sci. 21:99-103.

Matsumoto et al., Bioorg Med. Chem. Lett. 2000 Aug 21; 10(16):1857-1861.

Remy I, Wilson I A, and Michnick S W (1998). Erythropoietin receptor activation by a ligand-induced conformation change. Science 283:990.

Rozinov M N and Nolan G P (1998). Evolution of peptides that modulate the spectral qualities of bound, small-molecule fluorophores. Chemistry & Biology 5:713.

Sei-lida Y, Koshimoto H, Kondo S, and Tsuji A (2000). Real-time monitoring of in vitro transcriptional RNA synthesis using fluorescence resonance energy transfer. Nucleic Acids Res. 28:e59.

Sokol D L, Zhang X, Ponzy L, and Gewirtz A M (1998). Real time detection of DNA.RNA hybridization in living cells. Proc. Natl. Acad. Sci USA 95:11538.

Stripecke R, Oliveira C C, McCarthy J E G, and Hentze M W (1994). Proteins binding to 5′ untranslated region sites: a general mechanism for translational regulation of mRNAs in human and yeast cells. Mol. Cell. Biol. 14:5898.

Tan and Frankel Proc. Natl. Acad. Sci. 92 (1995) 5282-5286.

Tan et al., Cell 1993 Jun 4;73(5):1031-40.

Taylor D L (2000). Miniaturized cell array methods and apparatus for cell-based screening. U.S. Pat. No. 6,103,479.

Tsien R Y, Rink T J, Poenie M (1985). Measurement of cytosolic free Ca2+ in individual small cells using fluorescence microscopy with dual excitation wavelengths. Cell Calcium 6:145-157.

Tsuji A, Koshimoto H, Sato Y, Hirano M, Sei-lida Y, Kondo S, and Ishibashi K (2000). Direct observation of specific messenger RNA in a single living cell under a fluorescence microscope. Biophys. J. 78:3260.

Valegard K, Murray J B, Stonehouse N J, van den Worm S, Stockley P G, and Liljas L (1997). The three-dimensional structures of two complexes between recombinant MS2 capsids and RNA operator fragments reveal sequence-specific protein-RNA interactions. J. Mol. Biol. 270:724.

Wickens, M P and Parker R (1999). Tethered function assay for protein function. U.S. Pat. No. 5,985,575.

Zlokamik G, Negulescu P A, Knapp TE, Mere L, Burres N, Feng L, Whitney M, Roemer K, Tsien R Y (1998). Quantitation of transcription and clonal selection of single living cells with β-lactamase as reporter. Science 279:84.

I claim:

1. A method for quantifying expression of one or more target genes in living cells, comprising:
(a) providing cells that possess:
(1) at least a first fluorescently labeled RNA binding polypeptide, wherein the first fluorescently labeled RNA binding polypeptide comprises a first RNA binding domain; and
(2) at least a first target gene of interest, where the target gene has been modified to comprise one or more nucleic acid sequences encoding a first binding site for the first RNA-binding domain, wherein upon expression of the first target gene into a first target RNA, the first binding site is specifically bound by the first fluorescently labeled RNA-binding polypeptide;
(b) scanning the cells to obtain fluorescent signals from the first fluorescently labeled RNA binding polypeptide;
(c) determining fluorescent emission intensities from the first fluorescently labeled RNA binding polypeptide at two different wavelengths;
(d) calculating a ratio of the fluorescent emission intensities from the first fluorescently labeled RNA binding polypeptide at the two different wavelengths; and
(e) calculating a quantity of the first target RNA in the cells from the ratio.

2. The method of claim 1 wherein the first fluorescently labeled RNA binding polypeptide further comprises a nuclear export signal.

3. The method of claim 2 wherein the nuclear export signal comprises an amino acid sequence of the general formula:
XXXLXXLXL, where X is any amino acid (SEQ ID NO:30).

4. The method of claim 3 wherein the nuclear export signal comprises an amino acid sequence selected from the group consisting of:

| | |
|---|---|
| ALQKKLEELELDE; | (SEQ ID NO:19) |
| DLQKKLEELELDE; | (SEQ ID NO:20) |
| LPPLERLTL; | (SEQ ID NO:23) |
| LQQQLGQLTL; | (SEQ ID NO:25) |
| LDKLSVLTLS; and | (SEQ ID NO:27) |
| LWQFLLQLLLD. | (SEQ ID NO:28) |

5. The method of claim 2 wherein the nuclear export signal comprises an amino acid sequence selected from the group consisting of:

| | |
|---|---|
| DKERWEDVKEEMTSALATMRVDYE; | (SEQ ID NO:21) |
| WDRTFSLFQQLLQSSFVVE; | (SEQ ID NO:22) |
| LALKLAGLDI; | (SEQ ID NO:24) |
| LESNLRELQI; and | (SEQ ID NO:26) |
| LCQAFSKVILA. | (SEQ ID NO:29) |

6. The method of claim 2 wherein the first fluorescently labeled RNA binding polypeptide comprises a fluorophores pair selected from the group consisting of:
a) a donor/acceptor pair for fluorescence resonance energy transfer;
b) an excimer forming-pair; and
c) an exciplex-forming pair.

7. The method of claim 6 wherein the fluorophore pair is a donor/acceptor pair for fluorescence resonance energy transfer.

8. The method of claim 7 wherein the donor/acceptor pair is selected from the group consisting of:
fluorescein/rhodamine;
fluorescein/eosin;
fluorescein/erythrosine;
fluorescein/QSY-7;
fluorescein/ALEXA FLUOR® 54;
fluorescein/BODIPY®-TMR Cy3;
fluorescein/ALEXA FLUOR® 532;
ALEXA FLUOR® 488/rhodamine;
ALEXA FLUOR® 488/eosin;
ALEXA FLUOR® 488/erythrosine;
ALEXA FLUOR® 488/QSY-7;
ALEXA FLUOR® 488/ALEXA FLUOR® 54;
ALEXA FLUOR® 488/ BODIPY®-TMR Cy3;
ALEXA FLUOR® 488/ALEXA FLUOR® 532;
ALEXA FLUOR® 532 /ALEXA FLUOR® 546;
ALEXA FLUOR® 532/rhodamine;
ALEXA FLUOR® 350/ALEXA FLUOR® 430;
ALEXA FLUOR® 430/ALEXA FLUOR® 532;
ALEXA FLUOR® 430/eosin;
ALEXA FLUOR® 430/rhodamine; and
ALEXA FLUOR® 430/BODIPY®-TMR Cy3.

9. The method of claim 6 wherein the fluorophore pair is a excimer-forming pair.

10. The method of claim 9 wherein the excimer-forming pair is selected from the group consisting of:
a) a pyrene pair; and
b) a BODIPY-FL® pair.

11. The method of claim 6 wherein the fluorophore pair is a exciplex-forming pair.

12. The method of claim 11 wherein the exciplex-forming pair consists of anthracene and diethylaniline.

13. The method of claim 7 wherein the ratio of emission intensities comprise a ratio of emission intensity for the donor and an emission intensity for the acceptor.

14. The method of claim 9 wherein the ratio of the emission intensities comprises a ratio of an emission intensity of the excimer pair and an emission intensity of a monomer of either of the non-covalently interacting fluorophores.

15. The method of claim 11 wherein the ratio of the emission intensities comprises a ratio of an emission intensity of the exciplex pair and an emission intensity of a monomer of either of the non-interacting fluorophores.

16. The method of claim 2 wherein the first fluorescently labeled RNA binding polypeptides are membrane permeant.

17. The method of claim 16 wherein the RNA binding domain comprises an amino acid sequence selected from the group consisting of:

| | |
|---|---|
| TRQARRNRRRRWRERQR; | (SEQ ID NO:6) |
| {M/L)DAQTRRRERRAEKQAQWK; | (SEQ ID NO:8) |
| NAKTRRHERRRKLAIER; | (SEQ ID NO:10) |
| MPKTRRRPRRSQRKRP; and | (SEQ ID NO:12) |
| GRKKRRQRRRPPQ. | (SEQ ID NO:14) |

18. The method of claim 1 wherein the first RNA binding domain comprises an amino acid selected from the group consisting of

| | | |
|---|---|---|
| (i) | MDAQTRRRERRAEKQAQWKAANKG; | (SEQ ID NO:31) |
| (ii) | MDAQTRRRERRAEKQAQWKAANK; | (SEQ ID NO:32) |
| (iii) | MDAQTRRRERRAEKQAQWK; | (SEQ ID NO:33) |
| (iv) | MDAQTRRRERRAEKQAQWKA; | (SEQ ID NO:34) |
| (v) | MDAQTRRRERRAEKQAQWKAA; | (SEQ ID NO:35) |
| (vi) | MDAQTRRRERRAEKQAQWKAAN; | (SEQ ID NO: 36) |
| (vii) | LDAQTRRRERRAEKQAQWKAANKG; | (SEQ ID NO:37) |
| (viii) | LDAQTRRRERRAEKQAQWKAANK; | (SEQ ID NO:38) |
| (ix) | LDAQTRRRERRAEKQAQWK; | (SEQ ID NO:39) |
| (x) | LDAQTRRRERRAEKQAQWKA; | (SEQ ID NO:40) |
| (xi) | LDAQTRRRERRAEKQAQWKAA; and | (SEQ ID NO:41) |
| (xii) | LDAQTRRRERRAEKQAQWKAAN. | (SEQ ID NO:42) |

19. The method of claim 18, wherein the target RNA comprises a nucleotide sequence of the general formula NNGC(C/G)CUG(G/A)(G/A)(G/A)AAGGGCRR, wherein N is G or is absent and R is C or is absent (SEQ ID NO:9).

20. The method of claim 1 wherein the first RNA binding domain comprises an amino acid sequence TRQARRNRRRWRERQR (SEQ ID NO:6).

21. The method of claim 20 wherein the target RNA comprises a nucleotide sequence GGUCUGGGCGCAGCGCAAGCUGACGGUACA (SEQ ID NO:7).

22. The method of claim 1 wherein the first RNA binding domain comprises an amino acid sequence NAKTRRHERRRKLAIER (SEQ ID NO:10).

23. The method of claim 22 wherein the target RNA comprises a nucleotide sequence of GGUGCGCUGACAAAGCGCGCC (SEQ ID NO:11).

24. The method of claim 1 wherein the first RNA binding domain comprises an amino acid sequence MPKTRRRPRRSQRKRP (SEQ ID NO: 12).

25. The method of claim 24 wherein the target RNA comprises a nucleotide sequence of GGGCGCCGGUACGCAAGUACGACGGUACGCUCC (SEQ ID NO:13).

26. The method of claim 1 wherein the first RNA binding domain comprises an amino acid sequence of GRKKRRQRRRPPQ (SEQ ID NO:14).

27. The method of claim 26 wherein the target RNA comprises a nucleotide sequence selected from the group consisting of GGCCAGAUCUGAGCCU (SEQ ID NO:15) and GGGAGCUCUCUGGCC (SEQ ID NO:16)

28. The method of claim 2 wherein the first fluorescently labeled RNA binding polypeptide comprises an amino acid sequence selected from the group consisting of:

| | |
|---|---|
| RQIKIWFQNRRMKWKK; | (SEQ ID NO:1) |
| GALFLGWLGAAGSTMGAWSQPKKKRKV; | (SEQ ID NO:2) |
| AAVALLPAVLLALLAP; | (SEQ ID NO:3) |
| GWTLNSAGYLLKINLKALAALAKKIL; | (SEQ ID NO:4) |
| KLALKLALKALKAAIKLA; and | (SEQ ID NO:5) |

amino acid sequences of between 4 and 30 amino acids comprising between 4 and 12 arginine residues.

29. The method of claim 2 further comprising contacting a subset of the cells with one or more test compounds, and comparing the quantity of the target RNA in cells contacted with the one or more test compounds with the quantity of the target RNA in cells not contacted with the one or more test compound.

30. The method of claim 2 wherein the scanning comprises obtaining a visual representation of the fluorescent signals from the first fluorescently labeled RNA binding polypeptide.

31. The method of claim 2 further comprising determining the localization of the first fluorescently labeled RNA binding polypeptide within individual cells.

32. The method of claim 2 wherein the calculating fluorescent emission intensities from the first fluorescently labeled RNA binding polypeptide at two different wavelengths is performed at multiple time points.

33. The method of claim 2 wherein the cells comprise two or more distinct populations of cells, and wherein the calculating fluorescent emission intensities from the first fluorescently labeled RNA binding polypeptide at two different wavelengths is compared between the distinct cell populations.

34. A fluorescently labeled RNA binding polypeptide, comprising:
(a) a non-naturally occurring amino acid sequence comprising
(i) a nuclear export signal; and
(ii) an RNA binding domain, wherein the amino acid; and
(b) a fluorophore pair selected from the group consisting of
(i) a donor/acceptor pair for fluorescence resonance energy transfer;
(ii) an excimer forming fluorophore pair; and
(iii) an exciplex forming fluorophore pair.

35. The fluorescently labeled RNA binding polypeptide of claim 35 wherein the amino acid sequence comprising an RNA binding domain is membrane permeant.

36. The fluorescently labeled RNA binding polypeptide of claim 34 wherein the nuclear export signal comprises an amino acid sequence of the general formula
XXXLXXLXL, where X is any amino acid (SEQ ID NO:30).

37. The fluorescently labeled RNA binding polypeptide of claim 36 wherein the nuclear export signal comprises an amino acid sequence selected from the group consisting of:

| | |
|---|---|
| ALQKKLEELELDE; | (SEQ ID NO:19) |
| DLQKKLEELELDE; | (SEQ ID NO:20) |
| LPPLERLTL; | (SEQ ID NO:23) |
| LQQQLGQLTL; | (SEQ ID NO:25) |
| LDKLSVLTLS; and | (SEQ ID NO:27) |
| LWQFLLQLLLD. | (SEQ ID NO:28) |

38. The fluorescently labeled RNA binding polypeptide of claim 34 wherein the nuclear export signal comprises an amino acid sequence selected from the group consisting of:

| | |
|---|---|
| DKERWEDVKEEMTSALATMRVDYE; | (SEQ ID NO:21) |
| WDRTFSLFQQLLQSSFVVE; | (SEQ ID NO:22) |
| LALKLAGLDI; | (SEQ ID NO:24) |
| LESNLRELQI; and | (SEQ ID NO:26) |
| LCQAFSKVILA. | (SEQ ID NO:29) |

39. The fluorescently labeled RNA binding polypeptide of claim 35 wherein the RNA binding domain comprises an amino acid sequence selected from the group consisting of

| | |
|---|---|
| TRQARRNRRRRWRERQR; | (SEQ ID NO:6) |
| MDAQTRRRERRAEKQAQWKAAN; | (SEQ ID NO:8) |
| NAKTRRHERRRKLAIER; | (SEQ ID NO:10) |
| MPKTRRRPRRSQRKRP; and | (SEQ ID NO:12) |
| GRKKRRQRRRPPQ. | (SEQ ID NO:14) |

40. The fluorescently labeled RNA binding polypeptide of claim 39 wherein the fluorophore pair is a donor/acceptor pair for fluorescence resonance energy transfer, and wherein the donor/acceptor pair is selected from the group consisting of:
fluorescein/rhodamine;
fluorescein/eosin;
fluorescein/erythrosine;
fluorescein/QSY-7;
fluorescein/ALEXA FLUOR® 54;
fluorescein/BODIPY®-TMR Cy3;
fluorescein/ALEXA FLUOR® 532;
ALEXA FLUORT 488/rhodamine;
ALEXA FLUOR® 488/eosin;
ALEXA FLUOR® 488/erythrosine;
ALEXA FLUOR® 488/QSY-7;
ALEXA FLUOR® 488/ALEXA FLUOR® 54;
ALEXA FLUOR® 488/BODIPY®-TMR Cy3;
ALEXA FLUOR® 488/ALEXA FLUOR® 532;
ALEXA FLUOR® 532/ALEXA FLUOR® 546;
ALEXA FLUOR® 532/rhodamine;
ALEXA FLUOR® 350/ALEXA FLUOR® 430;
ALEXA FLUOR® 430/ALEXA FLUOR® 532;
ALEXA FLUOR® 430/eosin;
ALEXA FLUOR® 430/rhodamine; and
ALEXA FLUOR® 430/BODIPY®-TMR Cy3.

41. The fluorescently labeled RNA binding polypeptide of claim 39 wherein the fluorophore pair is an excimer-forming pair.

42. The fluorescently labeled RNA binding polypeptide of claim 41 wherein the excimer forming pair is selected from the group consisting of:
a) a pyrene pair; and
b) a BODIPY-FL® pair.

43. The fluorescently labeled RNA binding polypeptide of claim 39 wherein the fluorophore pair is an exciplex-forming pair.

44. The fluorescently labeled RNA binding polypeptide of claim 43 wherein the exciplex-forming pair consists of anthracene and diethylaniline.

45. The fluorescently labeled RNA binding polypeptide of claim 34, wherein the amino acid sequence further comprises an amino acid sequence selected from the group consisting of:

| | |
|---|---|
| RQIKIWFQNRRMKWKK; | (SEQ ID NO:1) |
| GALFLGWLGAAGSTMGAWSQPKKKRKV; | (SEQ ID NO:2) |
| AAVALLPAVLLALLAP; | (SEQ ID NO:3) |
| GWTLNSAGYLLKINLKALAALAKKIL; | (SEQ ID NO:4) |
| KLALKLALKALKAALKLA; and | (SEQ ID NO:5) |

amino acid sequences of between 4 and 30 amino acids comprising between 4 and 12 arginine residues.

46. A fluorescently labeled RNA binding polypeptide comprising:
(a) an RNA binding domain consisting of an amino acid sequence selected from the group consisting of:

| | | |
|---|---|---|
| (i) | MDAQTRRRERRAEKQAQWKAANKG; | (SEQ ID NO:31) |
| (ii) | MDAQTRRRERRAEKQAQWKAANK; | (SEQ ID NO:32) |
| (iii) | MDAQTRRRERRAEKQAQWK; | (SEQ ID NO:33) |
| (iv) | MDAQTRRRERRAEKQAQWKA; | (SEQ ID NO:34) |
| (v) | MDAQTRRRERRAEKQAQWKAA; | (SEQ ID NO:35) |
| (vi) | MDAQTRRRERRAEKQAQWKAAN; | (SEQ ID NO:36) |
| (vii) | LDAQTRRRERRAEKQAQWKAANKG; | (SEQ ID NO:37) |
| (viii) | LDAQTRRRERRAEKQAQWKAANK; | (SEQ ID NO:38) |
| (ix) | LDAQTRRRERRAEKQAQWK; | (SEQ ID NO:39) |
| (x) | LDAQTRRRERRAEKQAQWKA; | (SEQ ID NO:40) |
| (xi) | LDAQTRRRERRAEKQAQWKAA; and | (SEQ ID NO:41) |
| (xii) | LDAQTRRRERRAEKQAQWKAAN; and | (SEQ ID NO:42) |

(b) a donor/acceptor fluorophore pair selected from the group consisting of:
fluorescein/rhodamine;
fluorescein/eosin;
fluorescein/erythrosine;
fluorescein/QSY-7;
fluorescein/ALEXA FLUOR® 54;
fluorescein/BODIPY®-TMR Cy3;
fluorescein/ALEXA FLUOR® 532;
ALEXA FLUOR® 488/rhodamine;
ALEXA FLUOR® 488/eosin;
ALEXA FLUOR® 488/erythrosine;
ALEXA FLUOR® 488/QSY-7;
ALEXA FLUOR® 488/ALEXA FLUOR® 54;
ALEXA FLUOR® 488/ BODIPY®-TMR Cy3;
ALEXA FLUOR® 488/ALEXA FLUOR® 532;
ALEXA FLUOR® 532 /ALEXA FLUOR® 546;
ALEXA FLUOR® 532/rhodamine;
ALEXA FLUOR® 350/ALEXA FLUOR® 430;
ALEXA FLUOR® 430/ALEXA FLUOR® 532;
ALEXA FLUOR® 430/eosin;
ALEXA FLUOR® 430/rhodamine; and
ALEXA FLUOR® 430/BODIPY®-TMR Cy3.

47. A fluorescently labeled RNA binding polypeptide comprising:
(a) an RNA binding domain consisting of an amino acid sequence selected from the group consisting of:

| | | |
|---|---|---|
| (i) | MDAQTRRRERRAEKQAQWKAANKG; | (SEQ ID NO:31) |
| (ii) | MDAQTRRRERRAEKQAQWKAANK; | (SEQ ID NO:32) |
| (iii) | MDAQTRRRERRAEKQAQWK; | (SEQ ID NO:33) |
| (iv) | MDAQTRRRERRAEKQAQWKA; | (SEQ ID NO:34) |
| (v) | MDAQTRRRERRAEKQAQWKAA; | (SEQ ID NO:35) |
| (vi) | MDAQTRRRERRAEKQAQWKAAN; | (SEQ ID NO:36) |
| (vii) | LDAQTRRRERRAEKQAQWKAANKG; | (SEQ ID NO:37) |
| (viii) | LDAQTRRRERRAEKQAQWKAANK; | (SEQ ID NO:38) |
| (ix) | LDAQTRRRERRAEKQAQWK; | (SEQ ID NO:39) |
| (x) | LDAQTRRRERRAEKQAQWKA; | (SEQ ID NO:40) |
| (xi) | LDAQTRRRERRAEKQAQWKAA; and | (SEQ ID NO:41) |
| (xii) | LDAQTRRRERRAEKQAQWKAAN; and | (SEQ ID NO:42) |

(b) a nuclear export signal consisting of an amino acid selected from the group consisting of:
(SEQ ID NO:30)

| | |
|---|---|
| (i) | XXXLXXLXL, where X is any amino |
| | acid; |

(SEQ ID NO:19)

| | |
|---|---|
| (ii) | ALQKKLEELELDE; |

(SEQ ID NO:20)

| | |
|---|---|
| (iii) | DLQKKLEELELDE; |

(SEQ ID NO:23)

| | |
|---|---|
| (iv) | LPPLERLTL; |

(SEQ ID NO:25)

| | |
|---|---|
| (v) | LQQQLGQLTL; |

(SEQ ID NO:27)

| | |
|---|---|
| (vi) | LDKLSVLTLS; |

(SEQ ID NO:28)

| | |
|---|---|
| (vii) | LWQFLLQLLLD; |

(SEQ ID NO:21)

| | |
|---|---|
| (viii) | DKFRWEDVKEEMTSALATMRVDYE; |

(SEQ ID NO:22)

| | |
|---|---|
| (ix) | WDRTFSLFQQLLQSSFVVE; |

(SEQ ID NO:24)

| | |
|---|---|
| (x) | LALKLAGLDI; |

(SEQ ID NO:26)

| | |
|---|---|
| (xi) | LESNLRELQI; and |

(SEQ ID NO:29)

| | |
|---|---|
| (xii) | LCQAFSKVILA; and |

(c) a donor/acceptor fluorophore pair selected from the group consisting of:
fluorescein/rhodamine;
fluorescein/eosin;
fluorescein/erythrosine;
fluorescein/QSY-7;
fluorescein/ALEXA FLUOR® 54;
fluorescein/BODIPY®-TMR Cy3;
fluorescein/ALEXA FLUOR® 532;
ALEXA FLUOR® 488/rhodamine;
ALEXA FLUOR® 488/eosin;
ALEXA FLUOR® 488/erythrosine;
ALEXA FLUOR® 488/QSY-7;
ALEXA FLUOR® 488/ALEXA FLUOR® 54;
ALEXA FLUOR® 488/ BODIPY®-TMR Cy3;
ALEXA FLUOR® 488/ALEXA FLUOR® 532;
ALEXA FLUOR® 532/ALEXA FLUOR® 546;
ALEXA FLUOR® 532/rhodamine;
ALEXA FLUOR® 350/ALEXA FLUOR® 430;
ALEXA FLUOR® 430/ALEXA FLUOR® 532;
ALEXA FLUOR® 430/eosin;
ALEXA FLUOR® 430/rhodamine; and
ALEXA FLUOR® 430/BODIPY®-TMR Cy3.
